(19) United States
(12) Patent Application Publication
(10) Pub. No.: US 2008/0190419 A1
(43) Pub. Date: Aug. 14, 2008

(54) Inhalation Device

(76) Inventors: Allen John Pearson, Cambridgeshire (GB); Paul Kenneth Rand, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/908,801

(22) PCT Filed: Mar. 16, 2006
(86) PCT No.: PCT/GB2006/000936

§ 371 Date: Sep. 17, 2007

(30) Foreign Application Priority Data
Mar. 17, 2005 (GB) 0505542.1

(51) Int. Cl. A61M 11/00

(52) U.S. Cl. 128/200.23

(57) ABSTRACT

An inhaler for delivering medicament by inhalation, comprising: a canister which comprises a body which includes a base and a head and defines a chamber containing medicament, and a valve stem which extends from the body and from which medicament is in use delivered on actuation of the canister; and an actuator which comprises a housing receiving the canister and an actuating mechanism for actuating the canister, wherein the actuating mechanism comprises a loading member which is fitted to or comprised in the canister and includes a loading section which is located at a distance spaced from the base of the body of the canister and acted upon to drive the loading member from a first, rest position to a second, actuated position in which the canister is actuated to deliver medicament, and an actuating member which is provided to the base of the body of the canister and coupled to the loading section of the loading member, and operable by a user to actuate the canister to deliver medicament.

### FIELD OF THE INVENTION

The present invention relates to an actuator for an inhaler for administering medicament by inhalation and to an inhaler including the same. The invention is particularly, but not exclusively, concerned with an actuator for a pressurised metered dose inhaler (pMDI).

### BACKGROUND OF THE INVENTION

pMDIs are well known in the art of inhalation devices. It is therefore not necessary to describe the construction and operation of a pMDI other than in bare essentials.

A pMDI comprises a canister and an actuator housing. The housing is generally tubular, although this is not essential, and generally formed of a plastics material, for instance by moulding. The canister comprises an open-ended canister, typically made from a metal such as aluminium. The open end of the canister is sealingly capped by a metering valve assembly. The valve assembly typically includes a hollow dispensing member or valve stem which projects from the outlet or business end of the canister. The dispensing member is mounted for sliding movement relative to the canister between an extended position, to which the dispensing member is biased by a biasing mechanism in the valve assembly, typically a return spring, and a depressed position.

In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may for example be HFA-134a or HFA-227.

Movement of the dispensing member from the extended position to the depressed position results in a metered dose of the aerosol formulation being dispensed from the canister through the dispensing member. Typically, the metering valve assembly is provided with a metering chamber of defined volume. In the extended position of the dispensing member, the content of the canister is placed in fluid communication with the metering chamber through the dispensing member so that the metering chamber is filled with the aerosol formulation. When the dispensing member is depressed, the metering chamber is isolated from the canister inner volume and placed in fluid communication with the external environment through the dispensing member. Thus, the defined volume of the aerosol formulation in the metering chamber is discharged to the external environment via the dispensing member.

Such metering valve assemblies are well known in the art and can be obtained from inter alia Bespak Plc (King's Lynn, Norfolk, United Kingdom) and Valois S.A.S. (Le Neubourg, France).

The housing typically comprises an internal passageway having an open end. The canister is slidable into the internal passageway through the open end with the canister being inserted valve assembly first into the internal passageway. A stem block, which receives the dispensing member of the canister when the canister is received in the housing in a “rest position”, has a passageway with an inlet end for receiving the dispensing member and an outlet end, which faces a dispensing outlet of the housing, typically a mouthpiece or a nasal nozzle. The stem block holds the dispensing member stationary whereby depression of the canister to its rest position further into the housing to an “actuated position” causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister from the rest position to the actuated position. The inspiratory airflow produced by the patient entrains the metered dose of the medicinal aerosol formulation into the patient's respiratory tract. So, a pMDI of the type described above is a breath-coordinated inhaler.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or other respiratory conditions, in which the inhalation of foreign material may cause severe irritation.

Developments to pMDIs have included the provision of actuation indicators or dose counters therefor. Such a dose counter is described in PCT Patent Application Nos. WO-A-9856444 and WO-A-2004/001664 to Glaxo Group Limited. The dose counter is fixably secured on the valve assembly end of the canister and includes a display which denotes the number of metered doses of the medicament formulation dispensed from, or remaining in, the canister. The display of the dose counter is visible to the patient through a window provided in the housing. The display may be presented by a plurality of indicator wheels rotatably mounted on a common axle, each wheel having numerals displayed in series around the circumference.

Many actuators have been developed with a view to facilitating the delivery of medicament, examples of which are disclosed in U.S. Pat. No. 3,272,391, U.S. Pat. No. 3,272,392, U.S. Pat. No. 4,678,106, U.S. Pat. No. 5,899,365, U.S. Pat. No. 6,237,812 and WO-A-99/49917.

It is an aim of the present invention to provide an improved actuator for an inhaler for administering medicament by inhalation and an inhaler including the same.

### SUMMARY OF THE INVENTION

In one aspect the present invention provides an inhaler for delivering medicament by inhalation according to claim 1 hereof.

Preferably, the loading section of the loading member is located at the head of the body of the canister.

Preferably, the actuating mechanism further comprises a drive member which operably couples the loading section of the loading member and the actuating member.

More preferably, the drive member is pivotally mounted, such as to be pivoted on operation of the actuating member to engage the loading section of the loading member.

Yet more preferably, the loading member is located over the base of the body of the canister.

In one embodiment the loading section of the loading member comprises a substantially annular section.

Preferably, the loading member comprises a sleeve which fits about an outer peripheral surface of the body of the canister, an end section at one end of the sleeve which engages the base of the body of the canister, and the loading section at the other end of the sleeve.

More preferably, the sleeve of the loading member extends substantially to the head of the body of the canister.

Preferably, the actuating member is located over the base of the body of the canister, and comprises a sleeve which fits about an outer peripheral surface of the sleeve of the loading member, an actuating element disposed to one, distal end of the sleeve which engages the drive member such as to effect pivoting of the same, and an end section at the other end of the sleeve which is acted upon by the user in operating the actuating member.

In one embodiment the actuating element of the actuating member comprises a flange element.

Preferably, the drive member extends between opposite sides of the canister and is pivotally mounted at one of the opposite sides of the canister, and the actuating element of the actuating member is located at the other of the opposite sides of the canister.

Preferably, the housing includes a nozzle block which receives the valve stem of the canister.

Preferably, the housing includes an outlet through which the user in use inhales.

More preferably, the outlet is a mouthpiece.

The present invention also extends to the actuator of the inhaler of the invention.

Other aspects and features of the invention are set forth in the appended claims and the exemplary embodiments which will now be described with reference to the accompanying Figures of drawings.

### BRIEF DESCRIPTION OF THE DRAWINGS

FIG. 1 illustrates a perspective view of an inhaler in accordance with a first embodiment of the present invention;

FIG. 2 illustrates an exploded perspective view of the inhaler of FIG. 1;

FIG. 3 illustrates a fragmentary, part-sectional view of the inhaler of FIG. 1, where illustrated in the inoperative, rest configuration;

FIG. 4 illustrates a fragmentary, part-sectional view of the inhaler of FIG. 1, where illustrated in the actuated configuration;

FIG. 5 illustrates a part-exploded perspective view of an inhaler in accordance with a second embodiment of the present invention;

FIG. 6 illustrates a part-sectional view of an inhaler in accordance with a third embodiment of the present invention, where illustrated in the inoperative, rest configuration; and

FIG. 7 illustrates a part-sectional view of the inhaler of FIG. 6, where illustrated in the actuated configuration.

### DETAILED DESCRIPTION OF EXEMPLARY EMBODIMENTS

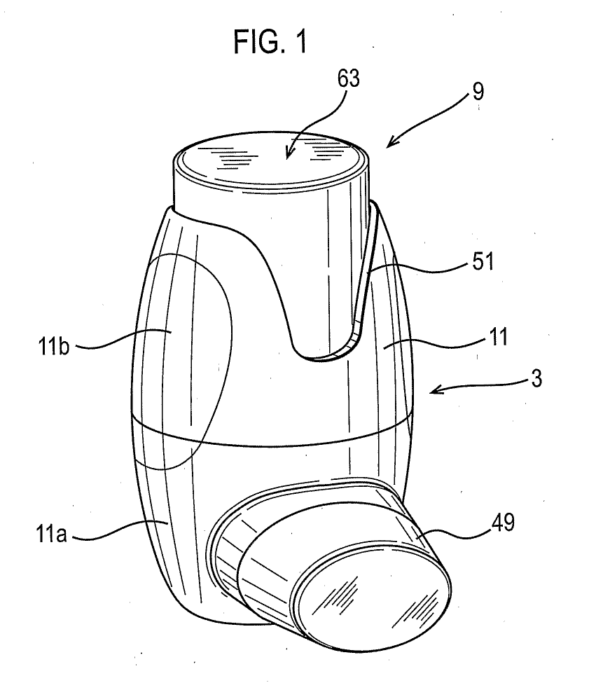

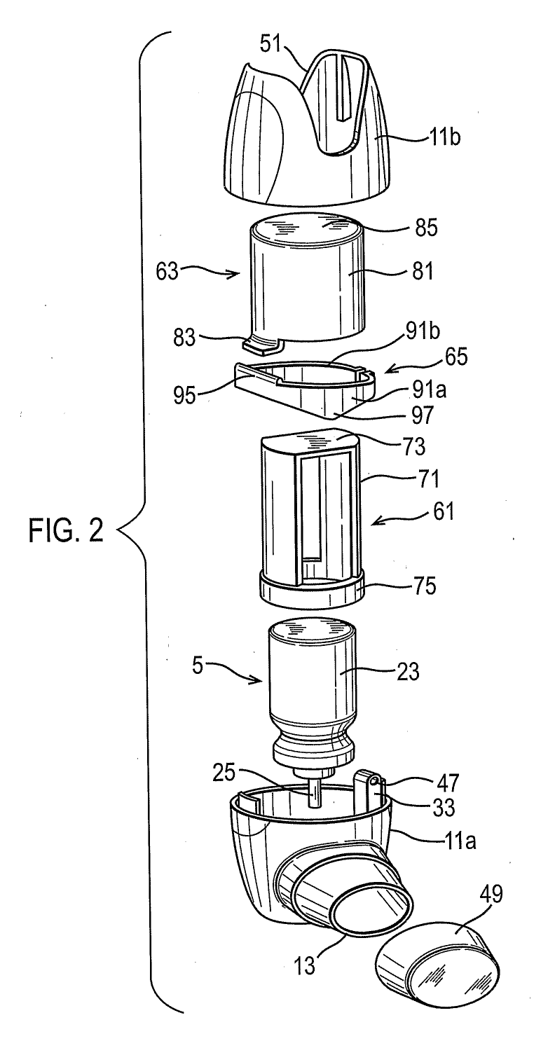

FIGS. 1 to 4 illustrate a hand-held, hand-operable inhaler of the pMDI type in accordance with a first embodiment of the present invention.

The inhaler comprises an actuator which comprises a main body 3, an aerosol canister 5 which is fitted in the main body 3 and contains medicament to be delivered on actuation of the inhaler, and an actuating mechanism 9 which is operable by a user to actuate the inhaler.

The main body 3 comprises a housing 11 in which the canister 5 is in use fitted, and a mouthpiece 13, in this embodiment a tubular element, which is in fluid communication with a lower end of the housing 11 and in use is gripped in the lips of the user. The mouthpiece 13 could instead be configured as a nasal nozzle.

The canister 5 in this embodiment is of standard type, as outlined supra, and comprises a body 23 which defines a chamber containing a medicament in a CFC-free propellant under pressure, for example an HFA propellant, a valve stem 25 which extends from one end, the head, of the body 23 and an internal metering valve (not illustrated) which is normally biased by an internal valve spring (not illustrated) to a closed position and opened to deliver a metered dose of medicament from the canister 5 when the valve stem 25 is depressed into the canister body 23.

In this embodiment the housing 11 comprises first and second housing parts 11a, b which are attached together, here by clips.

The first, lower housing part 11a is in fluid communication with the mouthpiece 13, and includes a nozzle block 31, in this embodiment disposed to a base surface of the first housing part 11a, for receiving the valve stem 25 of the canister 5, and a support member 33 to which a drive member 65 of the actuating mechanism 9 is pivotally coupled, as will be described in more detail hereinbelow. In this embodiment the lower housing part 11a is formed, here by moulding, as a single, integral unit.

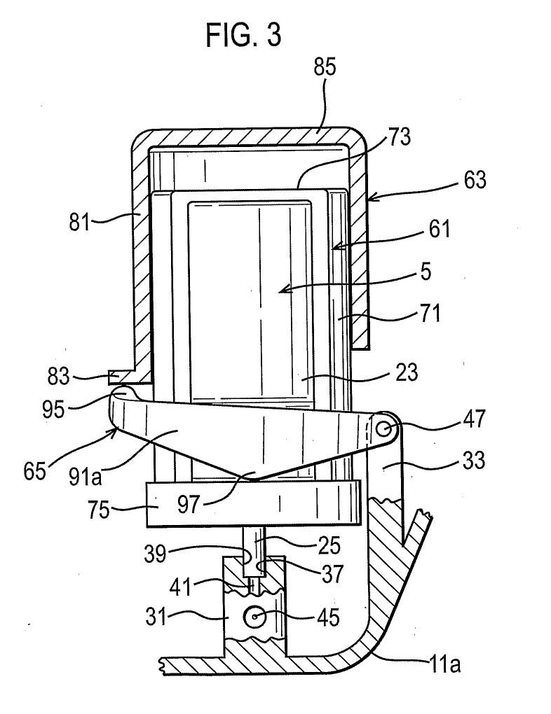

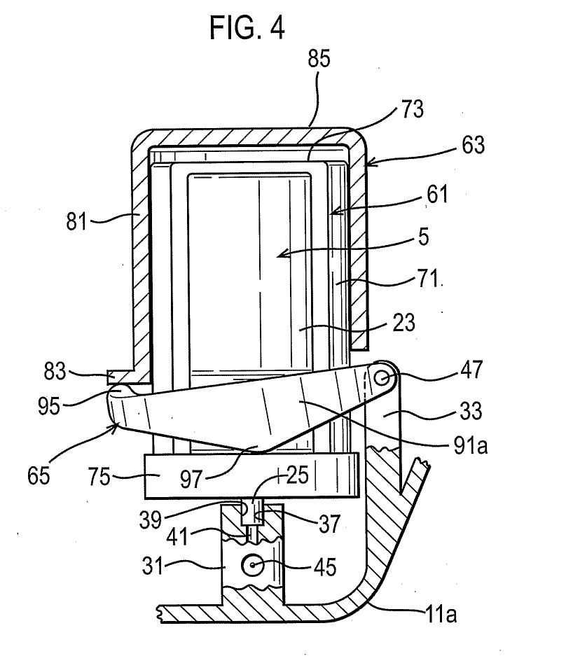

Referring particularly to FIGS. 3 and 4, the nozzle block 31 includes a tubular bore 37 for receiving the valve stem 25 of the canister 5, which in this embodiment is co-axial with the longitudinal axis of the housing 11. The tubular bore 37 is open at one, the upper, end thereof and includes an upper section 39 which has an internal dimension which is substantially the same as the outer dimension of the valve stem 25 and a lower section 41 which has a smaller dimension, which sections 39, 41 together define an annular seat for the distal end of the valve stem 25. The tubular bore 37 further includes a laterally-directed spray orifice 45 in the lower section 41 thereof which is configured to direct a spray into and through the mouthpiece 13.

Referring again particularly to FIGS. 3 and 4, in this embodiment the support member 33 is an upstanding member which includes a pivot 47 about which the drive member 65 of the actuating mechanism 9 is pivotally supported.

The inhaler further comprises a mouthpiece cap 49 which provides for closure of the mouthpiece 13.

The second, upper housing part 11a includes an aperture 51 at the upper end thereof, which allows for operation of the actuating mechanism 9, as will be described in more detail hereinbelow.

The actuating mechanism 9 comprises a loading member 61 which is fitted over the base of the body 23 of the canister 5, an actuating member 63 which is disposed at the aperture 51 in the second housing part 11b, and a drive member 65 which operably couples the loading member 61 and the actuating member 63, such as to provide for the loading member 61, and hence the body 23 of the canister 5, to be driven downwardly on depression of the actuating member 63.

In this embodiment the loading member 61 comprises a sleeve 71, here a part-tubular, substantially U-shaped sleeve, which is a close fit with the outer peripheral wall of the body 23 of the canister 5 and extends over substantially the entire length of the body 23 of the canister 5 from the base to the head of the body 23 of the canister 5, an end section 73 at one, the upper, end of the sleeve 71, here which spans the sleeve 71, which engages the base of the body 23 of the canister 5, and a loading section 75, here an annular flange, at the other, lower end of the sleeve 71, which is engaged by the drive member 65 to load the canister 5, as will be described in more detail hereinbelow.

In this embodiment the actuating member 63 is a cap element, here generally in the form of a button, which comprises a sleeve 81, here a tubular sleeve, which is a close fit with the outer peripheral wall of the sleeve 71 of the loading member 61 and includes an actuating element 83, here a flange element, at one, the lower, distal end of the sleeve 81 and to a side of the canister 5 opposite the support member 33, and an end section 85 at the other, upper end of the sleeve 81 which encloses the other end of the sleeve 81.

In this embodiment the drive member 65 comprises first and second drive arms 91a, b which are pivotally coupled at one end to the pivot 47 on the supporting member 33 and extend about opposed sides of the sleeve 71 of the loading member 61, such as to engage the loading section 75 of the loading member 61 at the opposed sides of the sleeve 71 of the loading member 61, and a connecting element 95 which interconnects the other, distal ends of the drive arms 91a, b at the side of the canister 5 opposite the support member 33 and is disposed such as to be engaged by the actuating element 83 of the actuating member 63, whereby, on depression of the actuating member 63, the drive arms 91a, b are driven in unison, here by pivoting about the pivot 47 on the support member 33, such as to drive the loading member 61, and hence the canister 5 downwardly to actuate the same.

In this embodiment the drive arms 91a, b each include a drive point 97, here defined by the junction of two inclined surfaces, which is located substantially in a plane which intersects the longitudinal axis of the housing 11, such that the canister 5 is loaded substantially uniformly along the longitudinal axis thereof and through the valve stem 25 of the canister 5.

Operation of the actuator will now be described hereinbelow.

The user first takes the actuator, as illustrated in FIG. 1, in one hand, and removes the mouthpiece cap 49.

The user then takes the mouthpiece 13 in his/her lips, and, in co-ordination with an inhalation breath, actuates the inhaler by depressing the actuating member 63 of the actuating mechanism 9 with one or more digits of the hand holding the actuator.

As illustrated in FIG. 4, depression of the actuating member 63 causes the actuating element 83 of the actuating member 63 to engage the connecting element 95 of the drive member 65, which in turn causes the drive member 65 to be pivoted about the pivot 47 on the support member 33 and drive the loading member 61, through engagement of the pivot points 97 of the respective drive arms 91a, b with the loading section 75 of the loading member 61.

This downward movement of the loading member 61 drives the body 23 of the canister 5 downwardly in relation to the stationary valve stem 25 of the canister 5, thus actuating the canister 5 to deliver a spray of the medicament formulation dispensed from the valve stem into and through the mouthpiece 13.

On releasing the actuating member 63, the inhaler is returned by the valve return spring to the rest configuration illustrated in FIG. 3, ready for subsequent actuation.

Following actuation, the inhaler is removed from the mouth, and the mouthpiece cap 49 is fitted to the mouthpiece, as illustrated in FIG. 1, ready for subsequent actuation.

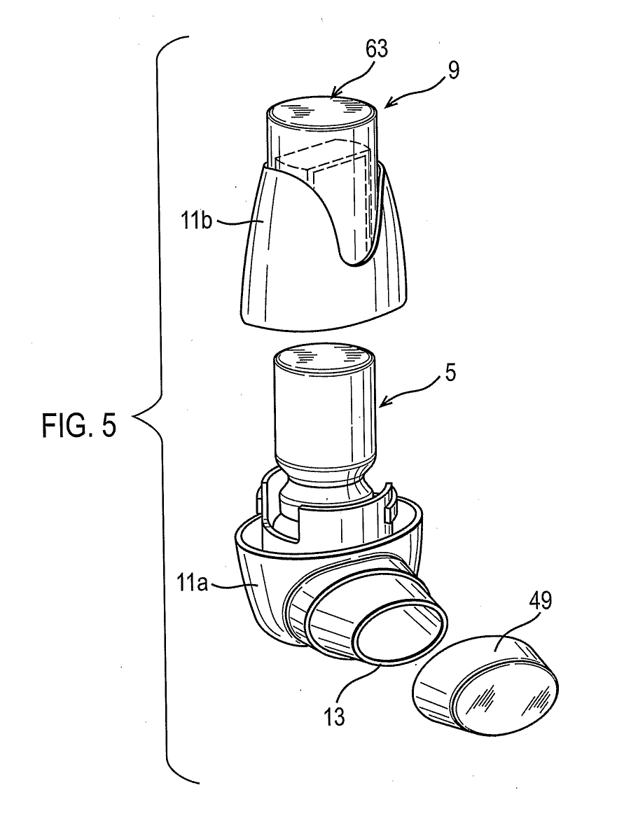

FIG. 5 illustrates a hand-held, hand-operable inhaler of the pMDI type in accordance with a second embodiment of the present invention.

The inhaler of this embodiment is very similar to the inhaler of the above-described embodiment, and thus, in order to avoid unnecessary duplication of description, only the differences will be described in detail, with like parts being designated by like reference signs.

The inhaler of this embodiment differs from that of the first-described embodiment in that the housing parts 11a, 11b are threadedly coupled, and the support member 33 is disposed to the upper housing part 11b. This configuration is advantageous in providing for ready replacement of the canister 5.

Operation of the inhaler of this embodiment is the same as for the above-described embodiment.

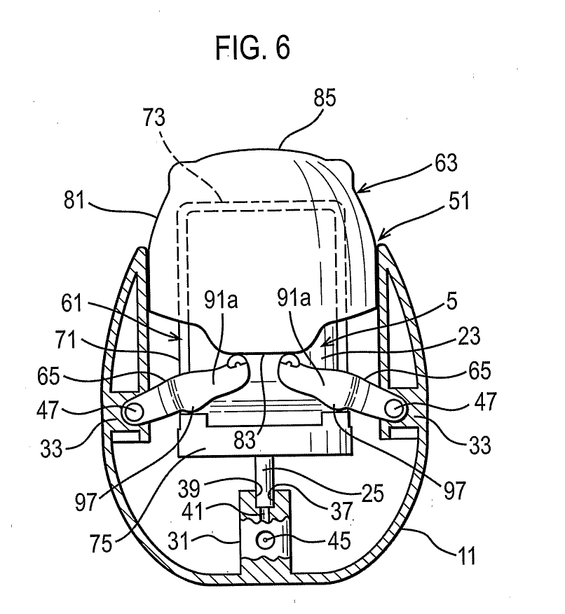

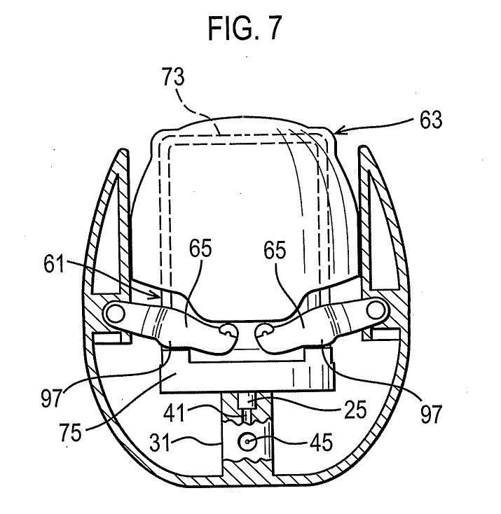

FIGS. 6 and 7 illustrate a hand-held, hand-operable inhaler of the pMDI type in accordance with a third embodiment of the present invention. The inhaler of this embodiment is very similar to the inhaler of the above-described first embodiment, and thus, in order to avoid unnecessary duplication of description, only the differences will be described in detail, with like parts being designated by like reference signs.

The inhaler of this embodiment differs from that of the first embodiment in the following noteworthy respects.

Firstly, the actuating mechanism 9 is provided with a pair of pivoting drive members 65 on opposing inner sides of the housing 11. Each drive member 65 has a pair of drive arms 91a, 91b (only one of which is shown), as in the first embodiment, although the distal ends of the drive arms 91a, 91b are not connected together in this embodiment. In fact, the drive members 65 are configured in a wishbone shape, with the drive arms 91a, 91b forming the limbs of the wishbone shape.

Secondly, the actuating member 63, again in the form of a cap, has a centrally disposed actuating element 83 on each opposing side thereof (only one shown), here in the form of a tongue at the distal end of the actuating member 63. Each actuating element 83 engages a pair of drive arms 91a, 91b, one from each drive member 65.

Operation of the inhaler of this embodiment is the same as for the above-described embodiment. More particularly, as shown in FIG. 7, depression of the actuating member 63 causes the actuating members 83 thereof to pivot the drive members 65 about the pivots 47 in a downward direction.

The loading member 61 is then driven downwardly through engagement of the pivot points 97 of the respective drive arms 91a, b with the loading section 75 of the loading member 61. This downward movement of the loading member 61 drives the body 23 of the canister 5 downwardly in relation to the stationary valve stem 25 of the canister 5, through engagement of the end section 73 with the base of the canister body 23, thus actuating the canister 5 to deliver a spray of the medicament formulation dispensed from the valve stem into and through the mouthpiece 13.

It will be appreciated that the actuating mechanisms in the illustrated embodiments of the invention provide a mechanical advantage. That is to say, the manual force required to be applied by the user to operate the inhaler (by overcoming the return force of the valve return spring) is less than would otherwise be the case, such as in operation of a standard pMDI where the user has to push down on the base of the canister 5 against the return force of the valve return spring. In the third embodiment, the use of two oppositely located drive members 65, compared to the single drive member 65 in the first embodiment, better balances and distributes the loading forces applied to the loading section 75.

Preferably, all of the parts of the actuator of the exemplary embodiments are made from a plastics material, for example by a moulding process.

The canister 5 in the illustrated embodiments may be furnished with a dose counter as described in WO-A-9856444 and WO-A-2004/001664, the entire contents of which are hereby incorporated herein by reference.

In a modification of the illustrated embodiments, not shown, the loading member 61 takes the form of an accessory which is fixedly connected to the head end of the canister 5 and which provides the loading section 75 for the drive member 65 to act on to move the canister 5 downwardly. As an example, the accessory may take the form of a dose counter as described in WO-A-9856444 and WO-A-2004/001664. The housing of such a dose counter may be adapted to present the loading section.

In an alternative modification of the illustrated embodiments, not shown, the loading section for the drive member 65 may be presented by a surface of the canister 5.

Finally, it will be understood that the present invention has been described in its exemplary embodiments and can be modified in many different ways without departing from the scope of the invention as defined by the appended claims.

Also, as regards the provision of reference signs in the appended claims, it is to be understood that reference signs are provided only for illustrative purposes and are not intended to confer any limitation to the claimed invention.

What is claimed is:

1. An inhaler for delivering medicament by inhalation, comprising:
a canister which comprises a body which includes a base and a head and defines a chamber containing medicament, and a valve stem which extends from the body and from which medicament is in use delivered on actuation of the canister; and
an actuator which comprises a housing receiving the canister, and an actuating mechanism for actuating the canister;
wherein the actuating mechanism comprises a loading member which is fitted to or comprised in the body of the canister and includes a loading section which is located at a distance spaced from the base of the body of the canister and acted upon to drive the loading member from a first, rest position to a second, actuated position in which the canister is actuated to deliver medicament, and an actuating member which is provided to the base of the body of the canister and coupled to the loading section of the loading member, and operable by a user to actuate the canister to deliver medicament.

2. The inhaler of claim 1, wherein the loading section of the loading member is located at the head of the body of the canister.

3. The inhaler of claim 1, wherein the actuating mechanism further comprises a drive member which operably couples the loading section of the loading member and the actuating member.

4. The inhaler of claim 3, wherein the drive member is pivotally mounted, such as to be pivoted on operation of the actuating member to engage the loading section of the loading member.

5. The inhaler of claim 4, wherein the loading member is located over the base of the body of the canister.

6. The inhaler of claim 5, wherein the loading section of the loading member comprises a substantially annular section.

7. The inhaler of claim 5, wherein the loading member comprises a sleeve which fits about an outer peripheral surface of the body of the canister, an end section at one end of the sleeve which engages the base of the body of the canister, and the loading section at the other end of the sleeve.

8. The inhaler of claim 7, wherein the sleeve of the loading member extends substantially to the head of the body of the canister.

9. The inhaler of claim 7, wherein the actuating member is located over the base of the body of the canister, and comprises a sleeve which fits about an outer peripheral surface of the sleeve of the loading member, an actuating element disposed to one, distal end of the sleeve which engages the drive member such as to effect pivoting of the same, and an end section at the other end of the sleeve which is acted upon by the user in operating the actuating member.

10. The inhaler of claim 9, wherein the actuating element of the actuating member comprises a flange element.

11. The inhaler of claim 9, wherein the drive member extends between opposite sides of the canister and is pivotally mounted at one of the opposite sides of the canister, and the actuating element of the actuating member is located at the other of the opposite sides of the canister.

12. The inhaler of claim 3, wherein the housing includes a nozzle block which receives the valve stem of the canister.

13. The inhaler of claim 3, wherein the housing includes an outlet through which the user in use inhales.

14. The inhaler of claim 13, wherein the outlet is a mouthpiece.

15-17. (canceled)
